(19) United States
(12) Patent Application Publication
(10) Pub. No.: US 2010/0112599 A1
(43) Pub. Date: May 6, 2010

(54) METHOD FOR THE COUPLED ENZYME IMMUNOCHEMICAL ASSAY OF ANALYTES BY MEANS OF ENDOGENOUS CALIBRATORS

(76) Inventors: Reinhard Renneberg, Hong Kong (CN); George W.H. Cautherley, Hong Kong (CN); Cangel P.Y. Chan, Hong Kong (CN); Matthias Lehmann, Berlin (DE); Karin Lehmann, Berlin (DE)

Correspondence Address: BUCHANAN INGERSOLL & ROONEY PC, P.O. BOX 1404, ALEXANDRIA, VA, 22313-1404, US

(21) Appl. No.: 12/299,926

(22) PCT Filed: May 7, 2007
(86) PCT No.: PCT/DE2007/000824

§ 371 Date: Dec. 21, 2009

(30) Foreign Application Priority Data
May 8, 2006 (DE) 10 2006 021 645.8

(51) Int. Cl. G01N 33/542

(52) U.S. Cl. 435/7.9

(57) ABSTRACT

The invention relates to a method for assaying a plurality of analytes such as e.g. metabolites and antigens in biological and other liquid samples by means of analytical elements, especially lateral-flow test strips, flow-through membrane systems (flow-through tests), wells/cavities of microtitre plates or test tubes, the method according to the invention being based on coupled enzyme and affinity reactions and being carried out by means of endogenous calibrators, i.e. endogenously produced substances, by means of which dilutions of sample matrices can be corrected (e.g. creatinine, glucose, glucose-6-phosphate, lactate, glutamate, aspartate, cholesterol, pyruvate, urea and triglycerides).

Application areas of the invention are principally medical diagnostics, the pharmaceutical industry and protection of the environment.

Preferably the invention concerns the simultaneous or sequential assay of antigens and metabolites. Within the meaning of the invention, these are principally high-molecular antigens, such as e.g. proteins or low-molecular haptenes, such as e.g. pesticides, neopterin, pollutants or hormones and in the case of metabolites e.g. glucose or creatinine.

The results can be determined directly from the assay with the aid of a nomogram, comparator (reference strips), reader or through comparison with the naked eye.

The invention relates to a method for assaying a plurality of analytes such as e.g. metabolites and antigens in biological and other liquid samples by means of analytical elements, especially lateral-flow test strips, flow-through membrane systems (flow-through tests), wells/cavities of microtitre plates or test tubes. Application areas of the invention are principally medical diagnostics, the pharmaceutical industry and protection of the environment.

Analytical elements, such as e.g. immuno-chromatographical test systems for assaying numerous analytes in biological liquids or for assaying pollutants in environmental samples have been known for years and have proved very successful in practice. They work mainly according to the sandwich or competition principle (in the case of small analytes). The use of affinity assays, e.g. immunoassays, receptor and DNA assays is becoming ever increasingly important for clinical and numerous other applications. In this case, very different analytes are assayed in different sample matrices (e.g. urine, saliva, tear fluid, sweat, liquor or blood). There is still one problem at the moment, that the dilution of at least part of the said matrices (e.g. urine) is not constant; therefore, in the course of the day, definite concentration changes occur, which influence the corresponding analyte measured value. However, endogenously formed substances are described for individual body matrices, with the help of which the corresponding dilution can be corrected; therefore, the system can be calibrated (Federal health sheet—health research—health protection 5 2005; Norpoth K, Heger M (1984), Creatinine as a reference variable for indicating substance concentrations in the urea. In: Hentschler D, Lehnert G (Hrsg) Biological Agent Tolerance Values (BAT values), occupational medical toxicological grounds, Vol. 1, Commission for the testing of health-hazardous biological agents of the DFG). Creatinine in the sample matrix urine is an example of these substances acting as endogenous calibrators. In the case of spontaneous urine samples of healthy test subjects, concentration fluctuations between 4 and 28 mmol of creatinine per litre of urine are quite normal (Fundamentals of Laboratory Testing: Urine, Roche Diagnostics GmbH, Mannheim, Germany). If these values are compared with the average value (15 mmol/l) of the 24 hour urine, it becomes clear that a dilution by the factor 3.5 is in the normal range, but the urine can also be concentrated by the factor 2. Based on an analyte to be determined in the sample matrix urine, this means: the measured value determined has to be corrected accordingly, so therefore a double determination (endogenous calibrator and analyte) followed by a calculation is necessary. In practice, this means carrying out two separate tests. In the case of urine, the creatinine concentration is determined first of all via an enzymatic cascade, and the volume of the analyte in the second stage (usually immuno-chemically). This approach is both time-consuming and labour-intensive.

Therefore, the object of the invention was to find new solutions for test systems that determine various analytes in sample matrices with concentrations that are not constant.

The invention is achieved according to the claims. It relates to a method for assaying a plurality of analytes such as e.g. metabolites and antigens in biological or other liquid samples by means of analytical elements, especially lateral-flow test strips, flow-through membrane systems (flow-through test), wells/cavities of microtitre plates or test tubes, the method being based on coupled enzyme and affinity reactions and implemented by means of endogenous calibrators.

The main idea consists of coupling enzyme and immunochemical methods and including the concentration of endogenous calibrators of the corresponding sample matrix in the total result, the analyte concentration being corrected automatically by the system achieved. With this combined enzymatic/immunological test system, body fluids are incubated with an enzyme mix (reaction 1), when the hydrogen peroxide (H₂O₂) produced with incubation through conversion of the endogenous calibrator is used partly or completely in a second, now immune reaction (reaction 2) with the same body fluid for signal formation through a marker enzyme. This signal is picked up and compared or offset with the signal of the H₂O₂ produced in the first reaction.

The two reactions of the method can be carried out simultaneously or sequentially and can also be carried out in one compartment or in 2 separate compartments. Preferably lateral-flow test strips, flow-through membrane systems (flow-through test), wells/cavities of microtitre plates or test tubes are used as compartments. According to the invention, the signal is picked up visually (naked eye), colourimetrically, through fluorescence or electrochemically.

The evaluation is made by means of nomogram, comparator (reference strip), reader or comparison with the naked eye, when, in the case of the latter, the number of signals generated enzymatically by means of a calibrator (e.g. test lines or test dots) is set in the ratio to the number of signals generated immunologically by means of analyte (e.g. test lines or test dots).

With the new method, body fluids, including urine, saliva, tear liquid, sweat, liquor or blood can be investigated. Creatinine, glucose, glucose-6-phosphate, lactate, glutamate, aspartate, cholesterol, pyruvate, urea and triglycerides in particular are used as endogenous calibrators. Marker enzymes are preferably peroxidases and oxidases. Preferably, an enzyme mix is used that reacts with the endogenous calibrator present in the body fluid concerned with the formation of H₂O₂. For example, in the case of the body fluid urine, when using creatinine as the endogenous calibrator, a mix of creatininase, creatinase and sarcosin oxidase is used. However, if glucose is used as the endogenous calibrator in the same sample matrix, glucose oxidase is used to generate the H₂O₂.

Surprisingly, it was found that with such a combined system in sample matrices with non-constant concentrations, a very wide range of analytes can be determined directly in a reliable manner, i.e. with the corresponding dilution correction. The invention is now explained in more detail below with the help of illustrations.

### EXAMPLES

### Example 1

### Assay of Cardio-Specific Fatty Acid Binding Protein (FABP) with Simultaneous Correction by Means of an Endogenous Calibrator (Creatinine) with Lateral-Flow Test Systems in the Sample Matrix Urine

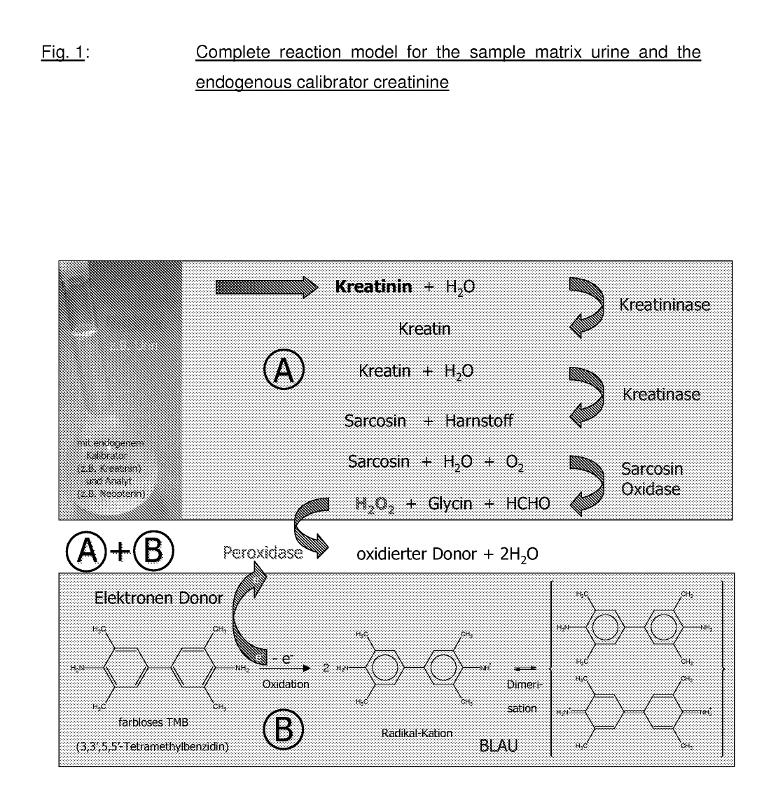

### See FIGS. 1, 3, 4 and 6

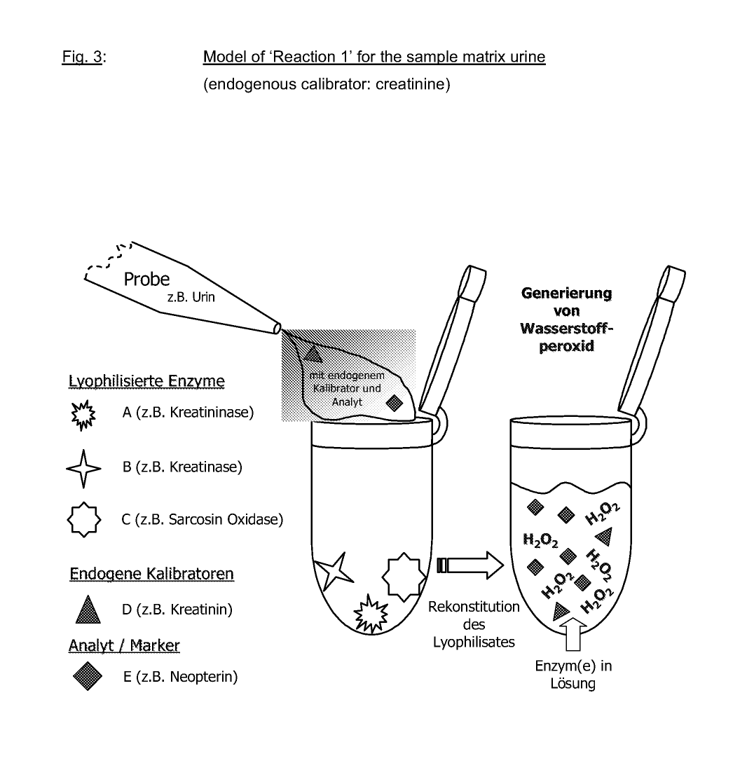

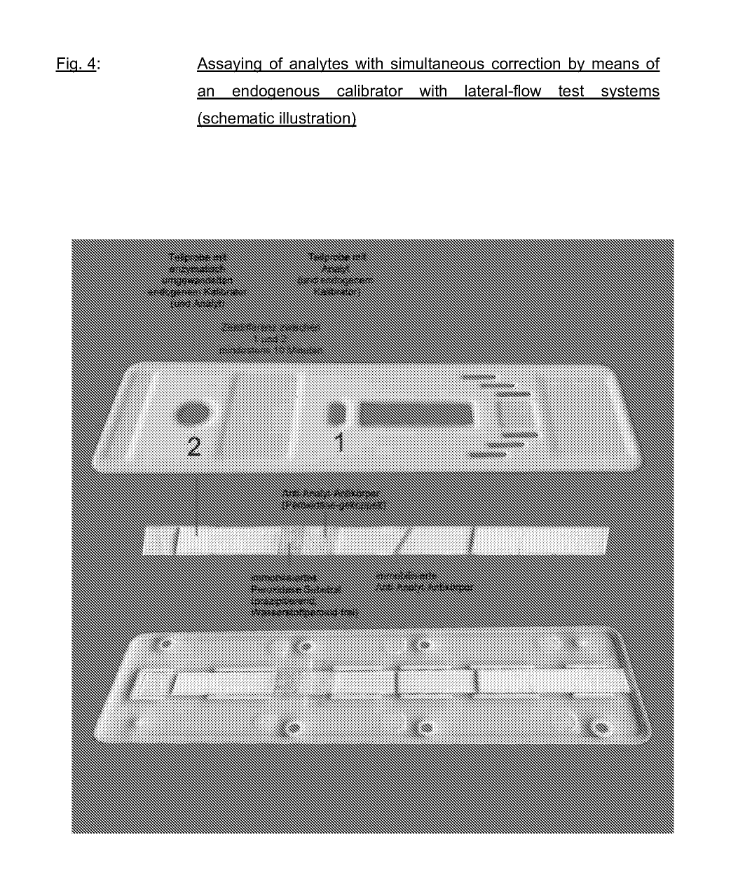

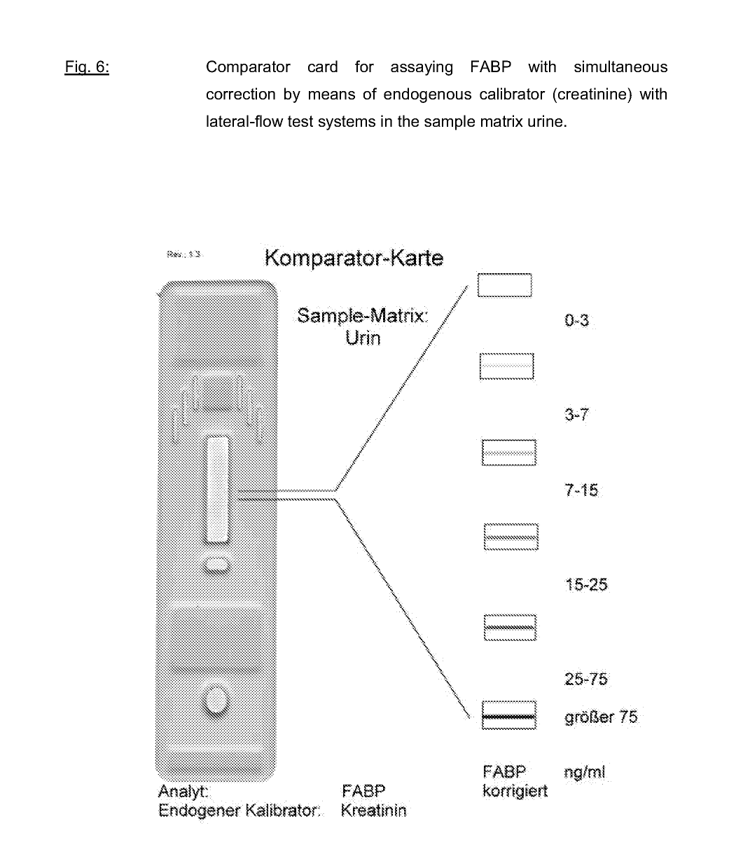

The urine sample (200 μl) is divided into 2 equal portions (A and B). Part A is introduced into a test tube (see FIG. 3), which contains the following enzyme mix in lyophilised form (per 20 μl): Creatininase (18.8 U/ml), Creatinase (7.5 U/ml) and Sarcosin Oxidase (11.3 U/ml). The test tube is agitated and incubated for 20 minutes at room temperature (20-25° C.). The endogenous creatinine present in the urine is converted by the enzyme mix reconstituted in this way to hydrogen peroxide (H₂O₂) among other things (see FIG. 1). Part B is diluted 1:4 first of all with a commercial protein stabiliser and 50 μl is then applied to the test strips (FIG. 4; sample opening 1). The anti-FABP/horse-radish peroxidase conjugate immobilised under sample opening 1 is dissolved out of its matrix, forms a complex (FABP-Anti-FABP/horse-radish peroxidase) with the analyte and in this form flows over the test field, on which a further anti-FABP antibody is immobilised as a catcher. With the presence of FABP, the latter binds the complex mentioned as a function of the analyte concentration. 20 minutes after the application of part B, 100 μl of pre-incubated part A is introduced into sample opening 2. Therefore, the immobilised H₂O₂-free and locally tightly limited precipitating peroxidase substrate on the test strip is dissolved and, together with the H₂O₂ (resulting from the conversion of the creatinine), flows over the test field. If the position of the catcher line is reached, the liquid, colourless substrate is now converted into a blue/violet precipitate. The volume of the precipitate, i.e. the generated signal, is a function of the analyte concentration and the volume of endogenous calibrator, which is used to generate the H₂O₂ necessary for the reaction. Highly endogenous calibrator (concentrated morning urine) generates a stronger signal than a sample with an average calibrator concentration (day-time urine) with the same analyte concentration. The test system described corrects this effect through the way it works and in this case, allows the true analyte concentration to be recorded with the aid of a comparator card (see FIG. 6).

### Example 2

### Assaying FABP with Simultaneous Correction by Means of an Endogenous Calibrator (Glucose) with Microtitre Plate-Based Macrodot Assays in the Sample Matrix Urine

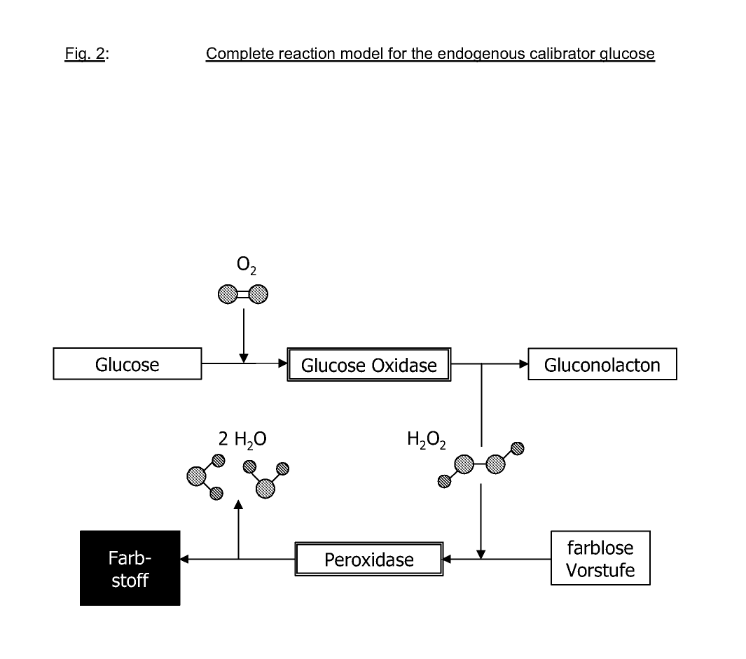

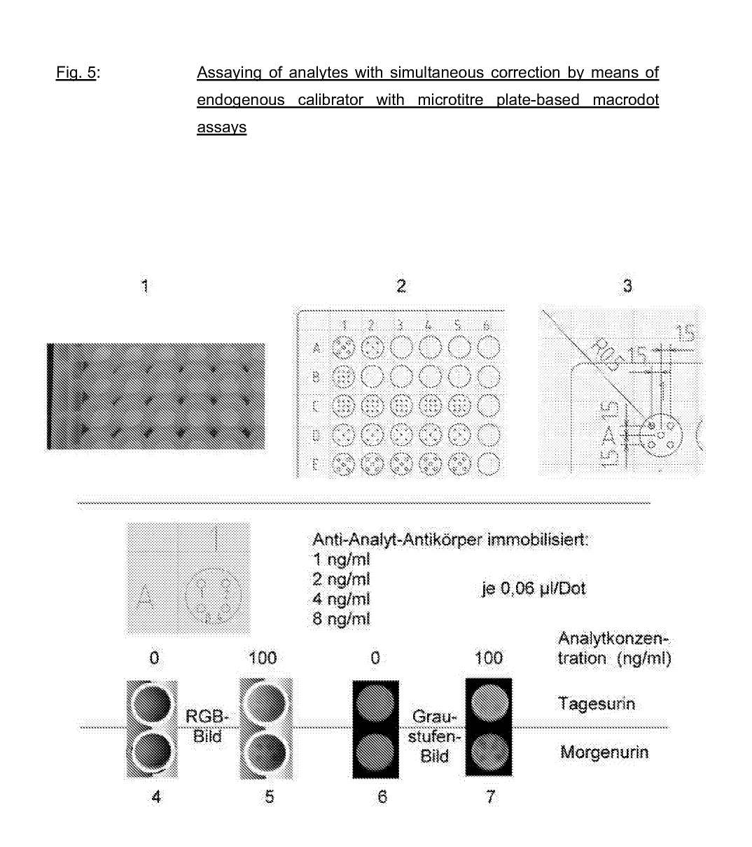

### See FIGS. 2 and 5

Commercial membrane-coated microtitre plates (96 wells) are used for the macrodot assays (FIG. 5/1). Preferably the membrane material consists of PVDF or nitrocellulose. The dot model used depends on the application concerned; examples are illustrated in FIG. 5/2. Preferably, 5 dots are used (FIG. 5/3), when the centre dot functions as a control dot as a rule. The outer dots can be used both for assaying different analytes and for the dilution corrections of individual analyte concentrations. The example to be described focuses on the assay of individual analytes (FABP) with simultaneous correction of the dilution of the sample matrix used (urine). 4 dots (see FIG. 5, centre section), which contain different concentrations of anti-FABP antibodies (catchers), were used. The urine sample (400 μl) is divided into 2 equal portions (A and B). Part A is introduced into a test tube containing 6.5 U glucose oxidase and 0.5 mM glucose. A small, fixed amount of glucose has to be added separately to produce a basic level of H₂O₂. The test tube is agitated and incubated for 60 minutes at room temperature (20-25° C.). The endogenous glucose present in the urine is converted to hydrogen peroxide (H₂O₂) among other things (see FIG. 2). Part B is diluted 1:10 first of all with a commercial protein stabiliser. The following reaction mixtures are made:

| | 1 | 2 |
|---|---|---|
| μl of the dilution (part B) | 199 | 194 |
| μl Anti-FABP antibody | 1 | 1 |
| horse-radish peroxidase conjugate | | |
| μl FABP from 4,000 ng/ml | 0 | 5 |
| solution | | |
| Corresponds to following | 0 | 100 |
| FABP final concentration in ng/ml | | |
| μl final volume | 200 | 200 |

100 μl reaction mixture is added to each well and incubated at room temperature (20-25° C.) every 60 minutes. FABP is added to be able to illustrate the effects to be described more clearly. Only with the presence of FABP can the following complex form on the catcher antibody: Catcher antibody/FABP/Anti-FABP antibody horse-radish peroxidase conjugate. The plate is then washed 4 times (0.1 M Na—P buffer, pH 7.2) and 50 μl of the following mixture is added to each well:

1 part Part A
1 part H₂O₂-free, locally tightly limited precipitating peroxidase substrate.

Incubation of at least 5 minutes at room temperature follows. In this case, at the positions at which the horse-radish peroxidase is bound, the liquid, colourless substrate is converted into a blue precipitate, when the volume of the precipitate, i.e. the signal generated, depends on the analyte concentration and the volume of endogenous calibrator (glucose) that is used to generate the H₂O₂ necessary for the reaction. The plate is then again washed 4 times (as above). After drying, the evaluation is made using an imaging process, for example. FIG. 5 shows both the RGB pictures without FABP addition (4) and with its addition (5) and also the grey stage pictures without FABP addition (6) and with it (7) used for the evaluation. Quantification took place on the basis of the latter pictures, whose data are given in Table 1. The effects of this self-calibrating assay become clear with the addition of 100 ng/ml FABP to both urine samples. It is evident from the last line that the morning urine used is concentrated 1.74 times compared with day-time urine. The signals generated through the model used with the catcher antibodies (1; 2; 4 and 8 ng/ml) are evaluated with reference to their dynamic. After strong signals for 1 ng/ml catcher antibody, from 2 ng/ml catcher antibody, a continuous signal rise to saturation takes place. With the same analyte concentration in the day-time urine, this saturation is reached very much earlier, because this non-concentrated sample contains less endogenous calibrator (glucose in this case) and therefore only a smaller amount of H₂O₂ can be formed. Therefore, the saturation curves can be described mathematically for diagnostically relevant concentration ranges and implemented in the evaluation software. The corrected concentration determination of analytes in samples then takes place through the comparison of the saturation curves.

TABLE 1
Quantification of the results shown in FIG. 5

| | without analyte addition | | | | with 100 ng/ml analyte addition | | | |
|---|---|---|---|---|---|---|---|---|
| Catcher Ng/ml | 1 | 2 | 4 | 8 | 1 | 2 | 4 | 8 |

Measured values correspond to the pixel average value of the range selected for evaluation

| | | | | | | | | |
|---|---|---|---|---|---|---|---|---|
| Day-time urine | 2,830 | 3,113 | 1,582 | 2,958 | 5,623 | 3,472 | 4,042 | 4,062 |
| Morning urine | 3,897 | 2,278 | 1,950 | 3,215 | 8,970 | 5,117 | 6,808 | 9,011 |
| Morning urine − Day-time urine | 1,067 | −835 | 368 | 257 | 3,347 | 1,645 | 2,766 | 4,949 |
| Morning urine:Day-time urine | 1.38 | 0.73 | 1.23 | 1.09 | 1.60 | 1.47 | 1.68 | 2.22 |

| | | |
|---|---|---|
| Total Day-time urine | 10,483 | 17,199 |
| Total Morning urine | 11,340 | 29,906 |
| Total morning urine − Total day-time urine | 857 | 12,707 |
| Total morning urine:Total day-time urine | 1.08 | 1.74 |

### Example 3

### Flow-Through Macro Dot Assays for Assaying Analytes with Simultaneous Correction by Means of Endogenous Calibrators for Various Sample Matrices

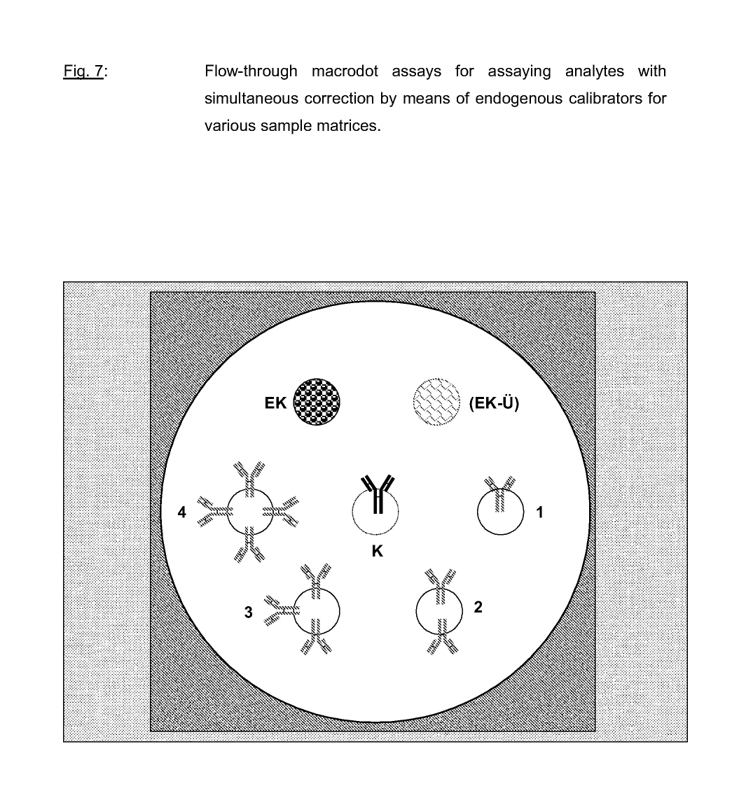

### FIG. 7

Example 3 describes the application of flow-though macrodot assays for assaying analytes with simultaneous correction by means of endogenous calibrators for various sample matrices. In this case, 7 dots are applied to a flow-through membrane system first of all (cf. FIG. 7). Dots ‘1 ’ to ‘4 ’ contain anti-analyte antibodies in increasing concentrations. Dot ‘K’ functions as a control dot and preferably contains anti-mouse immunoglobulin G or protein A. Dot ‘EK’ is used for assaying the corresponding endogenous calibrator and therefore preferably consists of individual enzymes or enzyme mixtures and also peroxidase. Dot ‘EK-Ü’ is optional and, when used, contains the corresponding calibrator (e.g. creatinine or glucose) in the optimum concentration for the application and also the components that are necessary for converting the calibrator into H₂O₂. A locally tightly limited precipitating peroxidase substrate is added to all the dots.

When this test system is used, the sample is pre-incubated first of all in a test tube that contains the necessary enzyme mix to convert the endogenous calibrator concerned or the corresponding individual enzyme and also horse-radish perioxidase-coupled anti-analyte antibodies. This reaction mix is then applied to the test field of the flow-through membrane system. If analyte is present in the sample, the analyte-horse-radish peroxidase-coupled anti-analyte antibody binds to the corresponding catcher antibodies (dots ‘1 ’ to ‘4 ’). Because of the different catcher antibody concentrations, a saturation takes place in the direction of dot ‘4 ’. The product formed through conversion of the endogenous calibrator in the reaction mix (preferably H₂O₂) now initiates the conversion of the H₂O₂-free, locally tightly limited precipitating peroxidase substrate previously immobilised in each dot from its colourless preliminary stage to a blue/violet precipitate. In this case, the precipitate volume depends on the peroxidase present locally and the H₂O₂ concentration, for example.

1. A method for performing a combined enzymatic/immunological test, comprising: (A) incubating at least one body fluid with an enzyme mix, and forming a product that is detected by a signal (B) using the product produced with the incubation through enzymatic conversion of an endogenous calibrator partly in a second, now immune reaction with the same body fluid for signal formation through a marker enzyme, and detecting this signal and comparing or offsetting it with the signal of the product produced in the first reaction.

2. The test method according to claim 1, wherein the two reactions are carried out simultaneously in parallel or sequentially.

3. The test method according to claim 1, wherein the two reactions are carried out in one compartment or in 2 separate compartments.

4. The test method according to claim 3, the compartments are selected from the group consisting of lateral-flow test strips, flow-through membrane systems, wells/cavities of microtitre plates and test tubes.

5. The test method according to claim 1, wherein the signal is picked up through a detection modality selected from the group consisting of visually, colourimetrically, turbidimetrically, through fluorescence and electro-chemically.

6. The test method according to claim 5, wherein the evaluation takes place through a modality selected from the group consisting of a nomogram, comparator, reader and comparison with the naked eye, and when, in the case of the latter, the number of signals generated enzymatically by means of a calibrator is set in the ratio to the number of signals generated immunologically by means of an analyte.

7. The test method according to claim 1, wherein the body fluid is one or more body fluid selected from the group consisting of urine, saliva, tear liquid, sweat, liquor, serum, plasma and blood.

8. The test method according to claim 1, wherein the endogenous calibrator is selected from the group consisting of creatinine, glucose, glucose-6-phosphate, lactate, glutamate, aspartate, cholesterol, pyruvate, urea, triglycerides, enzymes, and ions.

9. The test method according to claim 1, wherein peroxidases and oxidases are used as a marker enzyme.

10. The test method according to claim 1, wherein the enzyme mix is used reacts with the endogenous calibrator present in the body fluid concerned with the formation of H₂O₂.

11. The test method according to claim 1, wherein the endogenous calibrator used is added to the reaction mix in a constant, optimum concentration for the application concerned.

12. The test method according to claim 11, the body fluid is urine, a mix of creatininase, creatinase, sarcosin oxidase is used when using creatinine as the endogenous calibrator or the enzyme glucose oxidase when using glucose as the endogenous calibrator.
